(19) United States
(12) Patent Application Publication
(10) Pub. No.: US 2022/0362429 A1
(43) Pub. Date: Nov. 17, 2022

(54) OLFACTORY ENVIRONMENT MANAGEMENT SYSTEM

(71) Applicant: DEEPSCENT INC., Daejeon (KR)

(72) Inventors: Il-bong KWON, Seoul (KR); Ki-back JUNG, Busan (KR); Ha-yan CHOI, Daejeon (KR)

(21) Appl. No.: 17/771,002

(22) PCT Filed: Oct. 15, 2020
(86) PCT No.: PCT/KR2020/014106

§ 371 Date: Apr. 21, 2022

(30) Foreign Application Priority Data
Oct. 22, 2019 (KR) 10-2019-0131339

(51) Int. Cl. A61L 9/012; G01N 33/00

(52) U.S. Cl. CPC A61L 9/012; G01N 33/0004; A61L 2209/111; A61L 2209/13

(57) ABSTRACT

An olfactory environment management system includes a plurality of odor sensors, an aroma diffusing device, and a control server. Each of the plurality of odor sensors detects a chemical substance and determines a detected concentration of the chemical substance. The aroma diffusing device is equipped with first to n-th aroma capsules selected among a plurality of aroma capsules. The control server controls a combination of scents diffused from the aroma diffusing device. The aroma diffusing device determines an epicenter concentration of the chemical substance based on the detected concentration received from each of the plurality of odor sensors and transmits an identity of the chemical substance and the epicenter concentration to the control server. The control server individually controls an intensity of the emission of the aromatic substance of each of the first to n-th aroma capsules based on the identity of the chemical substance and the epicenter concentration.

### TECHNICAL FIELD

Example embodiments relate to an olfactory environment management system, and more particularly to an olfactory environment management system that detects a smell around the olfactory environment management system and diffuses scents suitable to remove the detected smell.

### BACKGROUND ART

Generally, smells diffusing through the air have great effect on people's lives.

For example, a good fragrance has the effect of making people feel better and refreshing people's mind. On the other hand, a bad smell has the effect of making people unpleasant and decreasing people's concentration and is even detrimental to health.

Therefore, maintaining a good olfactory environment in the indoor living space may help improve the quality of life.

To this end, various types of deodorization devices for removing a bad smell have been developed. Conventional deodorization devices remove a bad smell using a material that adsorbs a specific chemical causing the bad smell or using a perfume that emits a scent.

However, although effective deodorization methods differ depending on the type of a bad smell, conventional deodorization devices use a fixed deodorization method. As such, conventional deodorization devices have a difficulty in removing various types of bad smells.

### DISCLOSURE OF INVENTION

### Technical Problem

Example embodiments are to solve the problems of the conventional technology, and more particularly to provide an olfactory environment management system that detects a smell around the olfactory environment management system and diffuses a combination of scents suitable to remove the detected smell.

### Solution to Problem

Some example embodiments are directed to provide an olfactory environment management system. The olfactory environment management system includes a plurality of odor sensors, an aroma diffusing device, and a control server. The plurality of odor sensors are dispersedly arranged in an indoor space. Each of the plurality of odor sensors is configured to detect a chemical substance included in a surrounding space and to determine a detected concentration of the chemical substance. The aroma diffusing device is arranged in the indoor space and is equipped with first to n-th aroma capsules selected among a plurality of aroma capsules, which emit respective aromatic substances having different scents from each other. The control server is connected to the aroma diffusing device through an internet and is configured to control a combination of scents diffused from the aroma diffusing device. The aroma diffusing device determines an epicenter concentration of the chemical substance based on the detected concentration of the chemical substance received from each of the plurality of odor sensors and transmits an identity of the chemical substance and the epicenter concentration of the chemical substance to the control server. The control server individually controls an intensity of the emission of the aromatic substance of each of the first to n-th aroma capsules, which are installed in the aroma diffusing device, based on the identity of the chemical substance and the epicenter concentration of the chemical substance received from the aroma diffusing device.

### Advantageous Effects of Invention

According to example embodiments, the olfactory environment management system determines a kind of a smell generated in the indoor space and provides a combination of scents suitable to remove the determined smell in the indoor space, such that the olfactory environment management system can manage an olfactory environment of the user in a good state.

### BRIEF DESCRIPTION OF DRAWINGS

FIG. 1 is a diagram illustrating an olfactory environment management system according to example embodiments.

FIG. 2 is a diagram illustrating an example of an aroma diffusing device included in the olfactory environment management system of FIG. 1.

FIG. 3 is a diagram illustrating an example of a first database included in the control server of FIG. 1.

FIG. 4 is a diagram illustrating an example of a second database included in the control server of FIG. 1.

FIG. 5 is a diagram illustrating another example of a second database included in the control server of FIG. 1.

### BEST MODE FOR CARRYING OUT THE INVENTION

Various example embodiments will be described more fully with reference to the accompanying drawings, in which some example embodiments are shown. The present inventive concept may, however, be embodied in many different forms and should not be construed as limited to the embodiments set forth herein. Rather, these embodiments are provided so that this disclosure will be thorough and complete, and will fully convey the scope of the present inventive concept to those skilled in the art. Like reference numerals refer to like elements throughout this application.

It will be understood that, although the terms first, second, etc. may be used herein to describe various elements, these elements should not be limited by these terms. These terms are used to distinguish one element from another. For example, a first element could be termed a second element, and, similarly, a second element could be termed a first element, without departing from the scope of the present inventive concept. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments and is not intended to be limiting of the inventive concept. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, various example embodiments will be described with reference to the accompanying drawings.

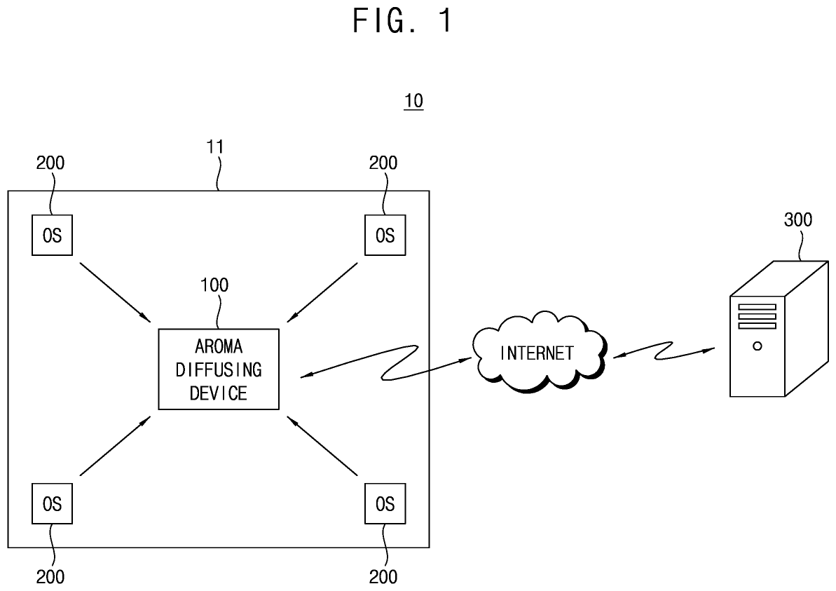

FIG. 1 is a diagram illustrating an olfactory environment management system according to example embodiments.

Referring to FIG. 1, an olfactory environment management system 10 includes an aroma diffusing device 100, a plurality of odor sensors OS 200, and a control server 300.

The aroma diffusing device 100 is arranged in an indoor space 11 in which a user lives.

The aroma diffusing device 100 may include a wireless communication module. The aroma diffusing device 100 may communicate with the control server 300 through a wireless communication using the wireless communication module.

As illustrated in FIG. 1, the wireless communication may correspond to an internet. In this case, the wireless communication module included in the aroma diffusing device 100 may correspond to a Wi-Fi communication module, and the aroma diffusing device 100 may be connected to the internet through an external wireless access point (AP) to communicate with the control server 300.

The control server 300 may control an operation of the aroma diffusing device 100 using the wireless communication.

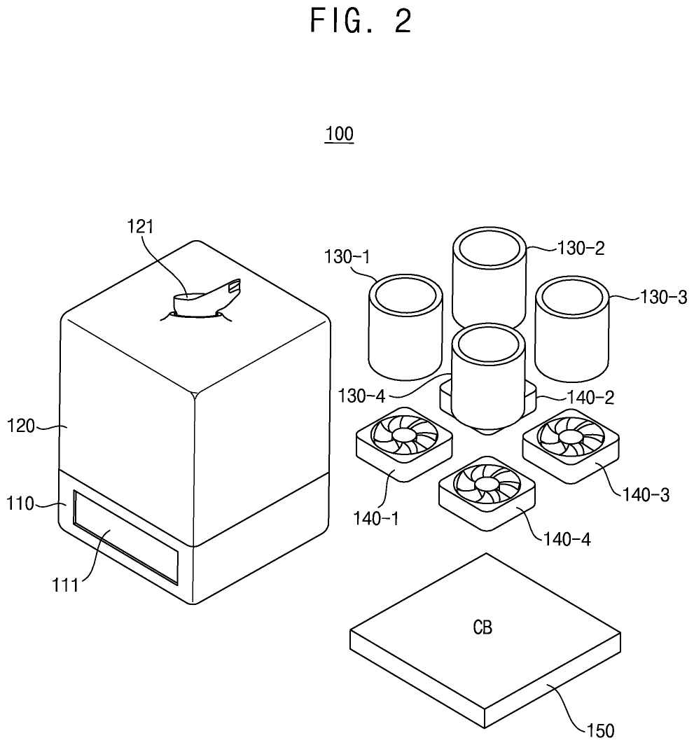

FIG. 2 is a diagram illustrating an example of an aroma diffusing device included in the olfactory environment management system of FIG. 1.

Referring to FIG. 2, the aroma diffusing device 100 may include a main body 110 and a housing 120.

In addition, the aroma diffusing device 100 may include first to n-th aroma capsules 130-1, 130-2, 130-3, and 130-4 that are installed on the main body 110 inside the housing 120. Here, n represents an integer equal to or greater than two.

The first to n-th aroma capsules 130-1, 130-2, 130-3, and 130-4 may emit respective aromatic substances having different scents from each other.

For example, a user may select n aroma capsules among a plurality of aroma capsules, which emit respective aromatic substances having different scents from each other, and the selected n aroma capsules may be installed on the main body 110 inside the housing 120 as the first to n-th aroma capsules 130-1, 130-2, 130-3, and 130-4.

According to the user's preference, each of the first to n-th aroma capsules 130-1, 130-2, 130-3, and 130-4 installed on the main body 110 inside the housing 120 may be replaced with another aroma capsule, which emits an aromatic substance of a different scent, among the plurality of aroma capsules.

In addition, the aroma diffusing device 100 may include first to n-th fans 140-1, 140-2, 140-3, and 140-4 that are installed on the main body 110 inside the housing 120.

The first to n-th fans 140-1, 140-2, 140-3, and 140-4 may be installed in areas adjacent to the first to n-th aroma capsules 130-1, 130-2, 130-3, and 130-4, respectively.

For example, as illustrated in FIG. 2, the first to n-th fans 140-1, 140-2, 140-3, and 140-4 may be installed under the first to n-th aroma capsules 130-1, 130-2, 130-3, and 130-4, respectively.

Therefore, as a rotation speed of each of the first to n-th fans 140-1, 140-2, 140-3, and 140-4 increases, an intensity of the emission of the aromatic substance of the first to n-th aroma capsules 130-1, 130-2, 130-3, and 130-4 may increase. On the other hand, as the rotation speed of each of the first to n-th fans 140-1, 140-2, 140-3, and 140-4 decreases, the intensity of the emission of the aromatic substance of the first to n-th aroma capsules 130-1, 130-2, 130-3, and 130-4 may decrease.

Although FIG. 2 illustrates that four aroma capsules 130-1, 130-2, 130-3, and 130-4 and four fans 140-1, 140-2, 140-3, and 140-4 are installed on the main body 110 inside the housing 120 as an example, example embodiments are not limited thereto. According to example embodiments, any number of aroma capsules and any number of fans may be installed on the main body 110 inside the housing 120.

In some example embodiments, the housing 120 may include a scent ventage 121.

For example, as illustrated in FIG. 2, the scent ventage 121 may be formed on the upper surface of the housing 120.

The aromatic substances emitted from the first to n-th aroma capsules 130-1, 130-2, 130-3, and 130-4 may be diffused to the outside of the aroma diffusing device 100 through the scent ventage 121 formed on the housing 120.

In some example embodiments, the aroma diffusing device 100 may include an electric motor connected to a lower part of the scent ventage 121 inside the housing 120.

In this case, the scent ventage 121 may be rotated based on an operation of the electric motor such that a direction of the scent ventage 121 may be changed.

Therefore, a direction in which the aromatic substances emitted from the first to n-th aroma capsules 130-1, 130-2, 130-3, and 130-4 are diffused to the outside of the aroma diffusing device 100 through the scent ventage 121 may be adjusted by controlling the electric motor.

In some example embodiments, the main body 110 may include a display device 111.

In this case, the aroma diffusing device 100 may display various information regarding the operation of the aroma diffusing device 100 on the display device 111.

A circuit board CB 150, which includes various kinds of electric modules, may be included in the main body 110.

For example, a controller, which controls an overall operation of the aroma diffusing device 100, the wireless communication module, which performs a wireless communication with the control server 300, a fan control module, which controls the rotation speed of each of the first to n-th fans 140-1, 140-2, 140-3, and 140-4, a motor control module, which controls the operation of the electric motor connected to the scent ventage 121, and a display driver, which controls an operation of the display device 111, may be mounted on the circuit board 150.

In the operation of the aroma diffusing device 100, the user may input first to n-th setting values, which correspond to the intensities of the emission of the aromatic substance of the first to n-th aroma capsules 130-1, 130-2, 130-3, and 130-4, respectively, in the aroma diffusing device 100.

In some example embodiments, each of the first to n-th setting values may correspond to a positive integer. For example, each of the first to n-th setting values may be a positive integer less than or equal to 100.

In this case, the aroma diffusing device 100 may individually control the intensities of the emission of the aromatic substances of the first to n-th aroma capsules 130-1, 130-2, 130-3, and 130-4 by adjusting the rotation speeds of the first to n-th fans 140-1, 140-2, 140-3, and 140-4 based on the first to n-th setting values, respectively.

Therefore, a combination of scents diffused from the aroma diffusing device 100 may be controlled precisely.

In some example embodiments, each of the first to n-th aroma capsules 130-1, 130-2, 130-3, and 130-4 may include an electronic tag storing a scent type of a corresponding aroma capsule. For example, the electronic tag may correspond to a radio frequency identification (RFID) tag.

In this case, the aroma diffusing device 100 may read first to n-th scent types from the electronic tags included in the first to n-th aroma capsules 130-1, 130-2, 130-3, and 130-4, respectively, and display the first to n-th scent types and the first to n-th setting values on the display device 111.

In some example embodiments, the user may remotely control the operation of the aroma diffusing device 100 using a mobile device connected to the aroma diffusing device 100.

In this case, the aroma diffusing device 100 may transmit the first to n-th scent types of the first to n-th aroma capsules 130-1, 130-2, 130-3, and 130-4, which are currently installed in the aroma diffusing device 100, to the mobile device, the user may transmit the first to n-th setting values, which correspond to the intensities of the emission of the aromatic substance of the first to n-th scent types, respectively, to the aroma diffusing device 100 using the mobile device, and the aroma diffusing device 100 may individually control the intensities of the emission of the aromatic substances of the first to n-th aroma capsules 130-1, 130-2, 130-3, and 130-4 by adjusting the rotation speeds of the first to n-th fans 140-1, 140-2, 140-3, and 140-4 based on the first to n-th setting values, respectively.

As described above, since the aroma diffusing device 100 according to example embodiments precisely controls the intensities of the emission of the aromatic substances of the first to n-th aroma capsules 130-1, 130-2, 130-3, and 130-4, the aroma diffusing device 100 may provide an accurate combination of scents that the user wants.

For example, whenever the aroma diffusing device 100 is operated based on the same first to n-th setting values, a combination of scents diffused from the aroma diffusing device 100 may be the same.

Although an example embodiment of the aroma diffusing device 100 is described above with reference to FIG. 2, a shape of the aroma diffusing device 100 illustrated in FIG. 2 is an example only and example embodiments are not limited thereto. According to example embodiments, the aroma diffusing device 100 may be formed in various shapes.

Referring again to FIG. 1, the plurality of odor sensors 200 may be dispersedly arranged in the indoor space 11 in which the user lives.

Although each of the plurality of odor sensors 200 is illustrated to be arranged at each corner of the indoor space 11 and the aroma diffusing device 100 is illustrated to be arranged at the center of the indoor space 11 in FIG. 2 as an example, example embodiments are not limited thereto. According to example embodiments, the plurality of odor sensors 200 and the aroma diffusing device 100 may be arranged at any location of the indoor space 11.

The aroma diffusing device 100 may be connected to each of the plurality of odor sensors 200 through a wireless communication or a wired communication.

In addition, the aroma diffusing device 100 may store in advance a relative position of each of the plurality of odor sensors 200 with respect to the aroma diffusing device 100.

In some example embodiments, the user may input the relative position of each of the plurality of odor sensors 200 with respect to the aroma diffusing device 100 in the aroma diffusing device 100.

Each of the plurality of odor sensors 200 may detect a chemical substance included in a surrounding space.

When detecting a chemical substance in the surrounding space, each of the plurality of odor sensors 200 may determine a detected concentration of the chemical substance and transmit an identity of the chemical substance and the detected concentration of the chemical substance to the aroma diffusing device 100.

The aroma diffusing device 100 may estimate an epicenter, which corresponds to a location at which the chemical substance is originated, and an epicenter concentration, which corresponds to a concentration of the chemical substance at the epicenter, based on the detected concentration of the chemical substance, which is received from each of the plurality of odor sensors 200, and the relative position of each of the plurality of odor sensors 200 with respect to the aroma diffusing device 100.

When the chemical substance having the epicenter concentration at the epicenter diffuses from the epicenter to the surrounding space, the detected concentration of the chemical substance, which is detected by the odor sensor 200, may be proportional to the epicenter concentration and be inversely proportional to a cube of a distance between the epicenter and the odor sensor 200.

Therefore, the aroma diffusing device 100 may calculate a relative position of the epicenter with respect to the aroma diffusing device 100 and the epicenter concentration to satisfy [Equation 1] for the detected concentration of the chemical substance, which is received from each of the plurality of odor sensors 200, and the relative position of each of the plurality of odor sensors 200 with respect to the aroma diffusing device 100.

$$\vert EC - Sj \vert = \sqrt[3]{\frac{G C}{Cj}} \qquad [ Equation 1 ]$$

Here, EC represents a position vector corresponding to the relative position of the epicenter with respect to the aroma diffusing device 100, j represents a positive integer, Sj represents a position vector corresponding to the relative position of the j-th odor sensor 200 with respect to the aroma diffusing device 100, GC represents the epicenter concentration, and Cj represents the detected concentration received from the j-th odor sensor 200.

Hereinabove, although the aroma diffusing device 100 is described to calculate the relative position of the epicenter with respect to the aroma diffusing device 100 and the epicenter concentration using the [Equation 1], example embodiments are not limited thereto. According to example embodiments, the aroma diffusing device 100 may calculate the relative position of the epicenter with respect to the aroma diffusing device 100 and the epicenter concentration using other equations, which model the relative position of the epicenter with respect to the aroma diffusing device 100, the epicenter concentration, the detected concentration of the chemical substance, which is received from each of the plurality of odor sensors 200, and the relative position of each of the plurality of odor sensors 200 with respect to the aroma diffusing device 100 in a different way from the [Equation 1].

The aroma diffusing device 100 may transmit the identity of the chemical substance and the epicenter concentration of the chemical substance to the control server 300.

The control server 300 may control a combination of scents diffused from the aroma diffusing device 100 through the wireless communication.

For example, the control server 300 may individually control the intensities of the emission of the aromatic substances of the first to n-th aroma capsules 130-1, 130-2, 130-3, and 130-4 based on the identity of the chemical substance and the epicenter concentration of the chemical substance, which are received from the aroma diffusing device 100, such that the aroma diffusing device 100 diffuses a combination of scents suitable to remove a smell of the chemical substance.

The control server 300 may include a first database 310 and a second database 320.

In some example embodiments, the first database 310 may store a bad smell name, a bad smell chemical substance corresponding to the bad smell name, and a threshold concentration of the bad smell chemical substance in association with each other.

Here, the threshold concentration represents a minimum concentration of the bad smell chemical substance at which a person starts to feel unpleasant due to a smell of the bad smell chemical substance.

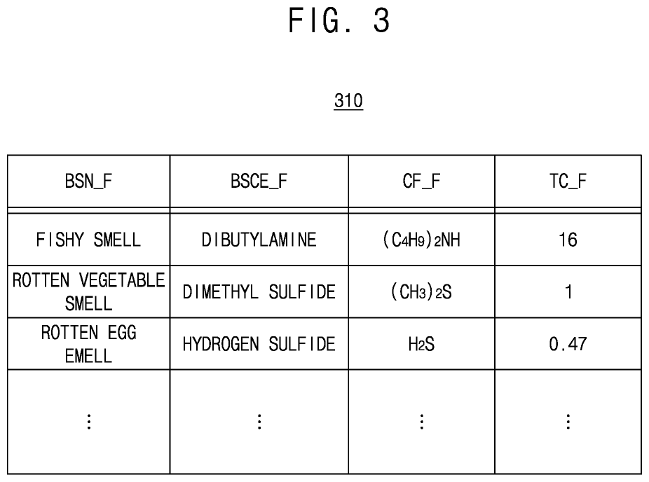

FIG. 3 is a diagram illustrating an example of a first database included in the control server of FIG. 1.

Referring to FIG. 3, the first database 310 may include a bad smell name field BSN_F, a bad smell chemical substance field BSCE_F, a chemical formula field CF_F storing a chemical formula of the bad smell chemical substance, and a threshold concentration field TC_F storing the threshold concentration of the bad smell chemical substance.

In FIG. 3, the first database 310 is illustrated to store a chemical substance, a chemical formula, and a threshold concentration for a fishy smell, a rotten vegetable smell, and a rotten egg smell as an example.

However, the first database 310 may store a bad smell name, a bad smell chemical substance corresponding to the bad smell name, and a threshold concentration of the bad smell chemical substance for other bad smells in association with each other.

In some example embodiments, the second database 320 may store a bad smell name, first to n-th deodorizing scent types suitable to remove a bad smell corresponding to the bad smell name, and first to n-th deodorizing setting values of the first to n-th deodorizing scent types in association with each other.

The bad smell names stored in the second database 320 may be the same as the bad smell names stored in the first database 310.

Therefore, records stored in the first database 310 and records stored in the second database 320 may be correlated with each other using the bad smell name as a key value.

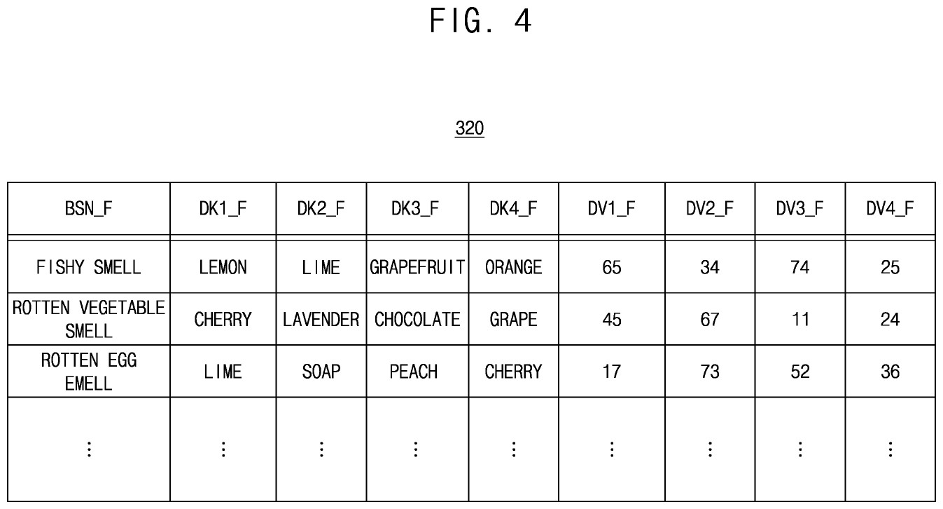

FIG. 4 is a diagram illustrating an example of a second database included in the control server of FIG. 1.

Referring to FIG. 4, the second database 320 may include a bad smell name field BSN_F, first to n-th deodorizing scent type fields DK1_F, DK2_F, DK3_F, and DK4_F, and first to n-th deodorizing setting value fields DV1_F, DV2_F, DV3_F, and DV4_F.

In FIG. 4, the second database 320 is illustrated to include first to fourth deodorizing scent type fields DK1_F, DK2_F, DK3_F, and DK4_F and first to fourth deodorizing setting value fields DV1_F, DV2_F, DV3_F, and DV4_F as an example.

In addition, in FIG. 4, the second database 320 is illustrated to store the first to n-th deodorizing scent types suitable to remove each of the fishy smell, the rotten vegetable smell, and the rotten egg smell that are stored in the first database 310, and the first to n-th deodorizing setting values of the first to n-th deodorizing scent types as an example.

Referring again to FIG. 1, when the control server 300 receives the identity of the chemical substance and the epicenter concentration of the chemical substance from the aroma diffusing device 100, the control server 300 may determine whether the chemical substance corresponding to the identity of the chemical substance received from the aroma diffusing device 100 is stored in the bad smell chemical substance field BSCE_F of the first database 310.

When the chemical substance corresponding to the identity of the chemical substance received from the aroma diffusing device 100 is not stored in the bad smell chemical substance field BSCE_F of the first database 310, the control server 300 may determine that the chemical substance corresponding to the identity of the chemical substance received from the aroma diffusing device 100 is not a smell-causing substance and ignore the identity of the chemical substance and the epicenter concentration of the chemical substance received from the aroma diffusing device 100.

On the other hand, when the chemical substance corresponding to the identity of the chemical substance received from the aroma diffusing device 100 is stored in the bad smell chemical substance field BSCE_F of the first database 310, the control server 300 may read the bad smell name and the threshold concentration, which correspond to the identity of the chemical substance, from the first database 310.

After that, the control server 300 may compare the epicenter concentration received from the aroma diffusing device 100 with the threshold concentration read from the first database 310.

When the epicenter concentration received from the aroma diffusing device 100 is smaller than or equal to the threshold concentration read from the first database 310, the control server 300 may determine that a concentration of the chemical substance diffused in the indoor space 11 is sufficiently low such that a person does not feel unpleasant and ignore the identity of the chemical substance and the epicenter concentration of the chemical substance received from the aroma diffusing device 100.

On the other hand, when the epicenter concentration received from the aroma diffusing device 100 is greater than the threshold concentration read from the first database 310, the control server 300 may read the first to n-th deodorizing scent types and the first to n-th deodorizing setting values, which correspond to the bad smell name read from the first database 310, from the second database 320.

After that, the control server 300 may transmit the first to n-th deodorizing scent types and the first to n-th deodorizing setting values, which are read from the second database 320, to the aroma diffusing device 100.

When the aroma diffusing device 100 receives the first to n-th deodorizing scent types and the first to n-th deodorizing setting values from the control server 300, the aroma diffusing device 100 may determine whether the first to n-th scent types of the first to n-th aroma capsules 130-1, 130-2, 130-3, and 130-4, which are currently installed in the aroma diffusing device 100, are the same as the first to n-th deodorizing scent types received from the control server 300.

When the first to n-th scent types of the first to n-th aroma capsules 130-1, 130-2, 130-3, and 130-4, which are currently installed in the aroma diffusing device 100, are the same as the first to n-th deodorizing scent types received from the control server 300, the aroma diffusing device 100 may adjust the direction of the scent ventage 121 in a direction toward the epicenter and individually control the intensities of the emission of the aromatic substances of the first to n-th aroma capsules 130-1, 130-2, 130-3, and 130-4 based on the first to n-th deodorizing setting values, respectively, which are received from the control server 300.

On the other hand, when the first to n-th scent types of the first to n-th aroma capsules 130-1, 130-2, 130-3, and 130-4, which are currently installed in the aroma diffusing device 100, are different from the first to n-th deodorizing scent types received from the control server 300, the aroma diffusing device 100 may display an aroma capsule replacement message.

In some example embodiments, the aroma diffusing device 100 may display the first to n-th deodorizing scent types received from the control server 300 together with the aroma capsule replacement message on the display device 111.

When the user replaces the first to n-th aroma capsules 130-1, 130-2, 130-3, and 130-4, which are currently installed in the aroma diffusing device 100, with the aroma capsules corresponding to the first to n-th deodorizing scent types, the aroma diffusing device 100 may read first to n-th scent types from the electronic tags included in the replaced first to n-th aroma capsules 130-1, 130-2, 130-3, and 130-4, respectively. When the aroma diffusing device 100 verifies that the read first to n-th scent types are the same as the first to n-th deodorizing scent types received from the control server 300, the aroma diffusing device 100 may adjust the direction of the scent ventage 121 in a direction toward the epicenter and individually control the intensities of the emission of the aromatic substances of the first to n-th aroma capsules 130-1, 130-2, 130-3, and 130-4 based on the first to n-th deodorizing setting values, respectively, which are received from the control server 300.

In other example embodiments, the second database 320 may store a bad smell name, first to n-th deodorizing scent types corresponding to each of all combinations of n aroma capsules selected among the plurality of aroma capsules, and first to n-th deodorizing setting values of the first to n-th deodorizing scent types suitable to remove a bad smell corresponding to the bad smell name in association with each other.

The bad smell names stored in the second database 320 may be the same as the bad smell names stored in the first database 310.

Therefore, records stored in the first database 310 and records stored in the second database 320 may be correlated with each other using the bad smell name as a key value.

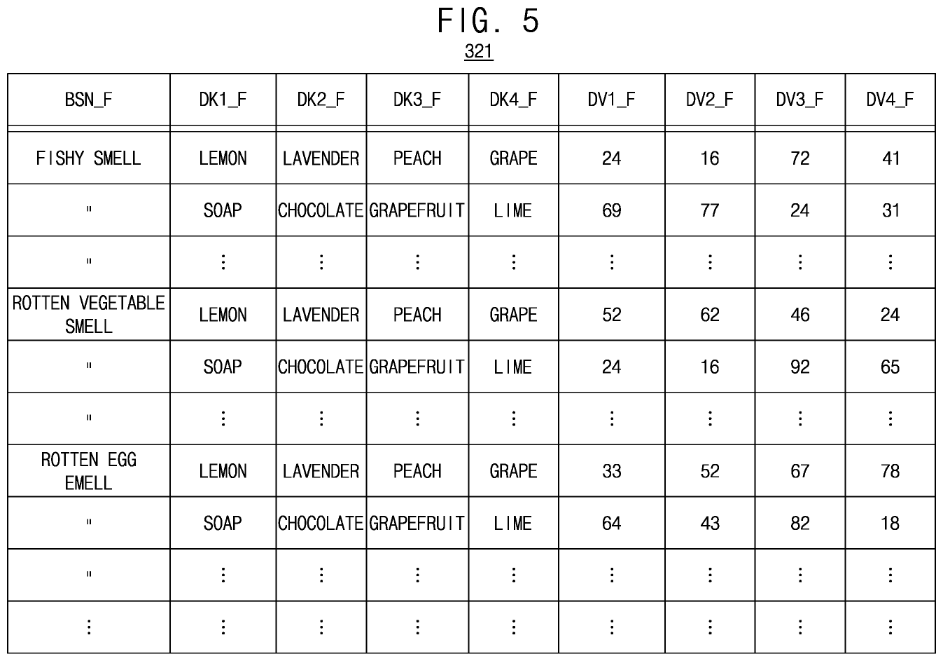

FIG. 5 is a diagram illustrating another example of a second database included in the control server of FIG. 1.

Referring to FIG. 5, the second database 320 may include a bad smell name field BSN_F, first to n-th deodorizing scent type fields DK1_F, DK2_F, DK3_F, and DK4_F, and first to n-th deodorizing setting value fields DV1_F, DV2_F, DV3_F, and DV4_F.

In FIG. 5, the second database 320 is illustrated to include first to fourth deodorizing scent type fields DK1_F, DK2_F, DK3_F, and DK4_F and first to fourth deodorizing setting value fields DV1_F, DV2_F, DV3_F, and DV4_F as an example.

In addition, in FIG. 5, the second database 320 is illustrated to store the first to n-th deodorizing scent types corresponding to each of all combinations of n aroma capsules selected among the plurality of aroma capsules, and the first to n-th deodorizing setting values of the first to n-th deodorizing scent types suitable to remove the bad smell for each of the fishy smell, the rotten vegetable smell, and the rotten egg smell that are stored in the first database 310 as an example.

As described above, each of the first to n-th aroma capsules 130-1, 130-2, 130-3, and 130-4 may include an electronic tag storing a scent type of a corresponding aroma capsule.

Therefore, when the first to n-th aroma capsules 130-1, 130-2, 130-3, and 130-4 are installed in the aroma diffusing device 100, the aroma diffusing device 100 may read first to n-th scent types from the electronic tags included in the first to n-th aroma capsules 130-1, 130-2, 130-3, and 130-4, respectively, and transmit the first to n-th scent types to the control server 300.

In the condition that the control server 300 includes the second database 320 illustrated in FIG. 5, when the control server 300 receives the identity of the chemical substance and the epicenter concentration of the chemical substance from the aroma diffusing device 100, the control server 300 may determine whether the chemical substance corresponding to the identity of the chemical substance received from the aroma diffusing device 100 is stored in the bad smell chemical substance field BSCE_F of the first database 310.

When the chemical substance corresponding to the identity of the chemical substance received from the aroma diffusing device 100 is not stored in the bad smell chemical substance field BSCE_F of the first database 310, the control server 300 may determine that the chemical substance corresponding to the identity of the chemical substance received from the aroma diffusing device 100 is not a smell-causing substance and ignore the identity of the chemical substance and the epicenter concentration of the chemical substance received from the aroma diffusing device 100.

On the other hand, when the chemical substance corresponding to the identity of the chemical substance received from the aroma diffusing device 100 is stored in the bad smell chemical substance field BSCE_F of the first database 310, the control server 300 may read the bad smell name and the threshold concentration, which correspond to the identity of the chemical substance, from the first database 310.

After that, the control server 300 may compare the epicenter concentration received from the aroma diffusing device 100 with the threshold concentration read from the first database 310.

When the epicenter concentration received from the aroma diffusing device 100 is smaller than or equal to the threshold concentration read from the first database 310, the control server 300 may determine that a concentration of the chemical substance diffused in the indoor space 11 is sufficiently low such that a person does not feel unpleasant and ignore the identity of the chemical substance and the epicenter concentration of the chemical substance received from the aroma diffusing device 100.

On the other hand, when the epicenter concentration received from the aroma diffusing device 100 is greater than the threshold concentration read from the first database 310, the control server 300 may read the first to n-th deodorizing setting values, which correspond to the bad smell name read from the first database 310 and the first to n-th scent types received from the aroma diffusing device 100, from the second database 320.

After that, the control server 300 may transmit the first to n-th deodorizing setting values, which are read from the second database 320, to the aroma diffusing device 100.

When the aroma diffusing device 100 receives the first to n-th deodorizing setting values from the control server 300, the aroma diffusing device 100 may adjust the direction of the scent ventage 121 in a direction toward the epicenter and individually control the intensities of the emission of the aromatic substances of the first to n-th aroma capsules 130-1, 130-2, 130-3, and 130-4 based on the first to n-th deodorizing setting values, respectively, which are received from the control server 300.

As described above with reference to FIGS. 1 to 5, each of the plurality of odor sensors 200 may detect the chemical substance included in the surrounding space and determine the detected concentration of the chemical substance. The aroma diffusing device 100 may estimate the epicenter, which corresponds to a location at which the chemical substance is originated, and the epicenter concentration, which corresponds to a concentration of the chemical substance at the epicenter, based on the detected concentration of the chemical substance, which is received from each of the plurality of odor sensors 200, and transmit the identity of the chemical substance and the epicenter concentration of the chemical substance to the control server 300.

In some example embodiments, the control server 300 may determine the first to n-th deodorizing scent types and the first to n-th deodorizing setting values of the first to n-th deodorizing scent types, which correspond to a combination of scents suitable to remove a bad smell caused by the chemical substance, based on the first database 310 and the second database 320 and transmit the first to n-th deodorizing scent types and the first to n-th deodorizing setting values to the aroma diffusing device 100. In other example embodiments, the control server 300 may determine the first to n-th deodorizing setting values, which are suitable to remove a bad smell caused by the chemical substance using the first to n-th aroma capsules 130-1, 130-2, 130-3, and 130-4 that are currently installed in the aroma diffusing device 100, based on the first database 310 and the second database 320 and transmit the first to n-th deodorizing setting values to the aroma diffusing device 100.

The aroma diffusing device 100 may adjust the direction of the scent ventage 121 in a direction toward the epicenter and individually control the intensities of the emission of the aromatic substances of the first to n-th aroma capsules 130-1, 130-2, 130-3, and 130-4 based on the first to n-th deodorizing setting values, respectively.

Therefore, the olfactory environment management system 10 according to example embodiments may determine a kind of a smell generated in the indoor space 11 and provide a combination of scents suitable to remove the determined smell in the indoor space 11, such that the olfactory environment management system 10 may be able to manage an olfactory environment of the user in a good state.

In some example embodiments, the control server 300 may further include a third database that cumulatively stores the identity of the chemical substance generated in the indoor space 11 and the user's choice of a combination of scents in association with each other.

As described above, the user may input first to n-th setting values, which correspond to the intensities of the emission of the aromatic substance of the first to n-th aroma capsules 130-1, 130-2, 130-3, and 130-4, respectively, in the aroma diffusing device 100.

Whenever the intensities of the emission of the aromatic substances of the first to n-th aroma capsules 130-1, 130-2, 130-3, and 130-4 are set to the first to n-th setting values, respectively, by the user's input, the aroma diffusing device 100 may transmit the first to n-th scent types of the first to n-th aroma capsules 130-1, 130-2, 130-3, and 130-4, respectively, and the first to n-th setting values together with the identity of the chemical substance and the epicenter concentration of the chemical substance to the control server 300.

In this case, the control server 300 may cumulatively store the identity of the chemical substance, the epicenter concentration of the chemical substance, the first to n-th scent types, and the first to n-th setting values in association with each other in the third database as a log data.

The aroma diffusing device 100 may include a scent recommendation function.

When the scent recommendation function is selected by the user, the aroma diffusing device 100 may estimate the epicenter, which corresponds to a location at which the chemical substance is originated, and the epicenter concentration, which corresponds to a concentration of the chemical substance at the epicenter, based on the detected concentration of the chemical substance, which is received from each of the plurality of odor sensors 200, and transmit a scent recommendation flag together with the identity of the chemical substance and the epicenter concentration of the chemical substance to the control server 300.

When the control server 300 receives the scent recommendation flag, the identity of the chemical substance, and the epicenter concentration from the aroma diffusing device 100, the control server 300 may determine first to n-th recommended scent types and first to n-th recommended setting values of the first to n-th recommended scent types by comparing the identity of the chemical substance and the epicenter concentration received from the aroma diffusing device 100 with the log data stored in the third database.

In some example embodiments, the control server 300 may read the log data, which includes the same identity of the chemical substance as the identity of the chemical substance received from the aroma diffusing device 100 and the epicenter concentration having a value within a predetermined ratio from the epicenter concentration received from the aroma diffusing device 100, from the third database.

After that, the control server 300 may determine first to n-th scent types, which correspond to a combination of scent types with a highest frequency among combinations of scent types included in the read log data, as the first to n-th recommended scent types.

In addition, the control server 300 may determine first to n-th values, which correspond to averages of the first to n-th setting values, respectively, for the first to n-th recommended scent types in the read log data, as the first to n-th recommended setting values.

For example, the control server 300 may determine a value, which correspond to an average of the setting values for the k-th recommended scent type in the read log data, as the k-th recommended setting value. Here, k represents a positive integer smaller than or equal to n.

After that, the control server 300 may transmit the first to n-th recommended scent types and the first to n-th recommended setting values to the aroma diffusing device 100.

When the aroma diffusing device 100 receives the first to n-th recommended scent types and the first to n-th recommended setting values from the control server 300, the aroma diffusing device 100 may determine whether the first to n-th scent types of the first to n-th aroma capsules 130-1, 130-2, 130-3, and 130-4, which are currently installed in the aroma diffusing device 100, are the same as the first to n-th recommended scent types received from the control server 300.

When the first to n-th scent types of the first to n-th aroma capsules 130-1, 130-2, 130-3, and 130-4, which are currently installed in the aroma diffusing device 100, are the same as the first to n-th recommended scent types received from the control server 300, the aroma diffusing device 100 may adjust the direction of the scent ventage 121 in a direction toward the epicenter and individually control the intensities of the emission of the aromatic substances of the first to n-th aroma capsules 130-1, 130-2, 130-3, and 130-4 based on the first to n-th recommended setting values, respectively, which are received from the control server 300.

On the other hand, when the first to n-th scent types of the first to n-th aroma capsules 130-1, 130-2, 130-3, and 130-4, which are currently installed in the aroma diffusing device 100, are different from the first to n-th recommended scent types received from the control server 300, the aroma diffusing device 100 may display an aroma capsule replacement message.

In some example embodiments, the aroma diffusing device 100 may display the first to n-th recommended scent types received from the control server 300 together with the aroma capsule replacement message on the display device 111.

When the user replaces the first to n-th aroma capsules 130-1, 130-2, 130-3, and 130-4, which are currently installed in the aroma diffusing device 100, with the aroma capsules corresponding to the first to n-th recommended scent types, the aroma diffusing device 100 may read first to n-th scent types from the electronic tags included in the replaced first to n-th aroma capsules 130-1, 130-2, 130-3, and 130-4, respectively. When the aroma diffusing device 100 verifies that the read first to n-th scent types are the same as the first to n-th recommended scent types received from the control server 300, the aroma diffusing device 100 may adjust the direction of the scent ventage 121 in a direction toward the epicenter and individually control the intensities of the emission of the aromatic substances of the first to n-th aroma capsules 130-1, 130-2, 130-3, and 130-4 based on the first to n-th recommended setting values, respectively, which are received from the control server 300.

As described above, the olfactory environment management system 10 according to example embodiments may cumulatively store the identity of the chemical substance generated in the indoor space 11 and the user's choice of a combination of scents in association with each other in the third database.

Therefore, when the olfactory environment management system 10 detects a smell in the indoor space 11, the olfactory environment management system 10 may determine a kind of the smell, determine a combination of scents using the third database that the user chose in the past when the determined smell was generated in the indoor space 11, and provide the combination of scents in the indoor space 11, such that the olfactory environment management system 10 may be able to manage an olfactory environment of the user in a good state.

Therefore, the olfactory environment management system 10 according to example embodiments may provide an increased satisfaction to the user.

The foregoing is illustrative of various example embodiments and is not to be construed as limited to the specific example embodiments disclosed and the figures attached, those skilled in the art will readily appreciate that many modifications are possible in the example embodiments without materially departing from the novel teachings and advantages of the present inventive concept, and the modifications to the disclosed example embodiments, as well as other example embodiments, are intended to be included within the scope of the appended claims.

### Description of Reference Numerals

10: an olfactory environment management system

11: an indoor space

100: an aroma diffusing device

110: a main body

111: display device

120: a housing

121: a scent ventage

130: an aroma capsule

140: a fan

150: a circuit board

200: an odor sensor

300: a control server

310: a first database

320: a second database

1. An olfactory environment management system, comprising:
a plurality of odor sensors dispersedly arranged in an indoor space, each of the plurality of odor sensors being configured to detect a chemical substance included in a surrounding space and to determine a detected concentration of the chemical substance;
an aroma diffusing device arranged in the indoor space, the aroma diffusing device being equipped with first to n-th aroma capsules selected among a plurality of aroma capsules, which emit respective aromatic substances having different scents from each other, n being an integer equal to or greater than two; and
a control server connected to the aroma diffusing device through an internet, the control server being configured to control a combination of scents diffused from the aroma diffusing device,
wherein the aroma diffusing device determines an epicenter concentration of the chemical substance based on the detected concentration of the chemical substance received from each of the plurality of odor sensors and transmits an identity of the chemical substance and the epicenter concentration of the chemical substance to the control server, and
wherein the control server individually controls an intensity of the emission of the aromatic substance of each of the first to n-th aroma capsules, which are installed in the aroma diffusing device, based on the identity of the chemical substance and the epicenter concentration of the chemical substance received from the aroma diffusing device.

2. The olfactory environment management system of claim 1, wherein the aroma diffusing device stores in advance a relative position of each of the plurality of odor sensors with respect to the aroma diffusing device and estimates an epicenter, which corresponds to a location at which the chemical substance is originated, and the epicenter concentration, which corresponds to a concentration of the chemical substance at the epicenter, based on the detected concentration of the chemical substance, which is received from each of the plurality of odor sensors, and the relative position of each of the plurality of odor sensors with respect to the aroma diffusing device.

3. The olfactory environment management system of claim 2, wherein the aroma diffusing device calculates a relative position of the epicenter with respect to the aroma diffusing device and the epicenter concentration to satisfy an equation
$$\vert EC - Sj \vert = \sqrt[3]{\frac{G C}{Cj}}$$
wherein EC represents a position vector corresponding to the relative position of the epicenter with respect to the aroma diffusing device, j represents a positive integer, Sj represents a position vector corresponding to the relative position of the j-th odor sensor with respect to the aroma diffusing device, GC represents the epicenter concentration, and Cj represents the detected concentration received from the j-th odor sensor.

4. The olfactory environment management system of claim 2, wherein the control server includes:
a first database configured to store a bad smell name, a bad smell chemical substance corresponding to the bad smell name, and a threshold concentration of the bad smell chemical substance in association with each other; and
a second database configured to store the bad smell name, first to n-th deodorizing scent types suitable to remove a bad smell corresponding to the bad smell name, and first to n-th deodorizing setting values of the first to n-th deodorizing scent types in association with each other.

5. The olfactory environment management system of claim 4, wherein, when the control server receives the identity of the chemical substance and the epicenter concentration from the aroma diffusing device, the control server reads the bad smell name and the threshold concentration, which correspond to the identity of the chemical substance, from the first database, and
wherein, when the epicenter concentration is greater than the threshold concentration read from the first database, the control server reads the first to n-th deodorizing scent types and the first to n-th deodorizing setting values, which correspond to the bad smell name, from the second database and transmits the first to n-th deodorizing scent types and the first to n-th deodorizing setting values to the aroma diffusing device.

6. The olfactory environment management system of claim 5, wherein, when first to n-th scent types of the first to n-th aroma capsules, which are installed in the aroma diffusing device, are the same as the first to n-th deodorizing scent types received from the control server, the aroma diffusing device adjusts a direction of a scent ventage, through which aromatic substances emitted from the first to n-th aroma capsules are diffused to the outside of the aroma diffusing device, in a direction toward the epicenter and individually controls the intensities of the emission of the aromatic substances of the first to n-th aroma capsules based on the first to n-th deodorizing setting values, respectively, which are received from the control server.

7. The olfactory environment management system of claim 5, wherein, when first to n-th scent types of the first to n-th aroma capsules, which are installed in the aroma diffusing device, are different from the first to n-th deodorizing scent types received from the control server, the aroma diffusing device displays an aroma capsule replacement message.

8. The olfactory environment management system of claim 2, wherein the control server includes:
a first database configured to store a bad smell name, a bad smell chemical substance corresponding to the bad smell name, and a threshold concentration of the bad smell chemical substance in association with each other; and
a second database configured to store the bad smell name, first to n-th deodorizing scent types corresponding to each of all combinations of n aroma capsules selected among the plurality of aroma capsules, and first to n-th deodorizing setting values of the first to n-th deodorizing scent types suitable to remove a bad smell corresponding to the bad smell name in association with each other.

9. The olfactory environment management system of claim 8, wherein each of the plurality of aroma capsules includes an electronic tag storing a scent type of a corresponding aroma capsule, and
wherein the aroma diffusing device reads first to n-th scent types from the electronic tags included in the first to n-th aroma capsules, respectively, and transmits the first to n-th scent types to the control server.

10. The olfactory environment management system of claim 9, wherein, when the control server receives the identity of the chemical substance and the epicenter concentration from the aroma diffusing device, the control server reads the bad smell name and the threshold concentration, which correspond to the identity of the chemical substance, from the first database, and
wherein, when the epicenter concentration is greater than the threshold concentration read from the first database, the control server reads the first to n-th deodorizing setting values, which correspond to the bad smell name read from the first database and the first to n-th scent types received from the aroma diffusing device, from the second database and transmits the first to n-th deodorizing setting values to the aroma diffusing device.

11. The olfactory environment management system of claim 10, wherein the aroma diffusing device adjusts a direction of a scent ventage, through which aromatic substances emitted from the first to n-th aroma capsules are diffused to the outside of the aroma diffusing device, in a direction toward the epicenter and individually controls the intensities of the emission of the aromatic substances of the first to n-th aroma capsules based on the first to n-th deodorizing setting values, respectively, which are received from the control server.

12. The olfactory environment management system of claim 2, wherein, when the intensities of the emission of the aromatic substances of the first to n-th aroma capsules are set to first to n-th setting values, respectively, by a user's input, the aroma diffusing device transmits first to n-th scent types of the first to n-th aroma capsules, respectively, and the first to n-th setting values together with the identity of the chemical substance and the epicenter concentration of the chemical substance to the control server, and
wherein the control server includes a third database that stores the identity of the chemical substance, the epicenter concentration of the chemical substance, the first to n-th scent types, and the first to n-th setting values in association with each other as a log data.

13. The olfactory environment management system of claim 12, wherein, when a scent recommendation function is selected by the user, the aroma diffusing device transmits a scent recommendation flag together with the identity of the chemical substance and the epicenter concentration of the chemical substance to the control server, and
wherein, when the control server receives the scent recommendation flag, the identity of the chemical substance, and the epicenter concentration from the aroma diffusing device, the control server determines first to n-th recommended scent types and first to n-th recommended setting values of the first to n-th recommended scent types by comparing the identity of the chemical substance and the epicenter concentration received from the aroma diffusing device with the log data stored in the third database, and transmits the first to n-th recommended scent types and the first to n-th recommended setting values to the aroma diffusing device.

14. The olfactory environment management system of claim 13, wherein the control server reads the log data, which includes the same identity of the chemical substance as the identity of the chemical substance received from the aroma diffusing device and the epicenter concentration having a value within a predetermined ratio from the epicenter concentration received from the aroma diffusing device, from the third database, determines first to n-th scent types, which correspond to a combination of scent types with a highest frequency among combinations of scent types included in the read log data, as the first to n-th recommended scent types, and determines first to n-th values, which correspond to averages of the first to n-th setting values, respectively, for the first to n-th recommended scent types in the read log data, as the first to n-th recommended setting values.

15. The olfactory environment management system of claim 13, wherein, when first to n-th scent types of the first to n-th aroma capsules, which are installed in the aroma diffusing device, are the same as the first to n-th recommended scent types received from the control server, the aroma diffusing device adjusts a direction of a scent ventage, through which aromatic substances emitted from the first to n-th aroma capsules are diffused to the outside of the aroma diffusing device, in a direction toward the epicenter and individually controls the intensities of the emission of the aromatic substances of the first to n-th aroma capsules based on the first to n-th recommended setting values, respectively, which are received from the control server.

16. The olfactory environment management system of claim 13, wherein, when first to n-th scent types of the first to n-th aroma capsules, which are installed in the aroma diffusing device, are different from the first to n-th recommended scent types received from the control server, the aroma diffusing device displays an aroma capsule replacement message.
